(19) United States
(12) Patent Application Publication
(10) Pub. No.: US 2026/0232203 A1
(43) Pub. Date: Aug. 13, 2026

(54) METHODS AND SYSTEMS FOR DETECTION OF VITAL SIGNS

(71) Applicant: ALGORIZED SÀRL, Etoy (CH)

(72) Inventors: Natalia LOPAREVA, Florel (Lavaux) (CH); Marc KUHN, Gockhausen (CH); Matthias LUDWIG, Weinfelden (CH)

(73) Assignee: ALGORIZED SÀRL, Etoy (CH)

(21) Appl. No.: 19/139,917

(22) PCT Filed: Oct. 17, 2023
(86) PCT No.: PCT/EP2023/078801

§ 371 Date: Jun. 17, 2025

(51) Int. Cl. A61B 5/0205; A61B 5/00; G01S 7/285; G01S 7/41; G01S 13/02; G01S 13/04; G01S 13/86; G01S 13/88

(52) U.S. Cl. CPC A61B 5/0205; A61B 5/0077; A61B 5/725; A61B 5/7257; A61B 5/742; A61B 5/746; G01S 7/285; G01S 7/411; G01S 13/0209; G01S 13/04; G01S 13/867; G01S 13/886

(57) ABSTRACT

Disclosed are systems and methods for detecting vital signs. We describe a method for detecting a presence of one or more living objects by measuring one or more vital signs of the one or more living objects, the method comprising: transmitting, by a transceiver, a first signal; receiving, by the transceiver, a response to the first signal from any object in an environment from which at least a part of the transmitted first signal has been reflected off from the object towards the transceiver; translating the response into raw data; selecting a bin from the raw data by processing and filtering the raw data based on a reflected energy received by the transceiver via the response; sampling results from the bin obtained via the selection; analyzing the results to determine the presence and one or more vital signs of any living object from which the transmitted first signal has been reflected off towards the transceiver; outputting one or more vital signs parameters corresponding to the determined one or more vital signs.

### FIELD OF THE INVENTION

The present invention generally relates to methods involving a signal input from a device for remotely detecting one or more vital signs (e.g. one or more of breathing rate, heart rate, blood flow) and/or behavior patterns (e.g. one or more of body position, motion speed, distance, and more) of living objects (people, animals), even through obstacles and clothing. This system and method, which can be applied to stationary or portable sensors without requiring on-body wearables or identifying tags, aim to provide real-time preventative alerts to potentially save lives. The methods comprise transmitting and receiving a first signal, and finding a peak of a power spectral density of the received signal. The system utilizes a device, whether stationary or mobile, configured to detect people and their behavior by analyzing signals from these devices using algorithms and classifiers, enabling accurate vital sign readings and age detection. The devices comprise a transceiver configured to transmit and receive a first signal, and a processor for finding a peak of the power spectral density of the received signal and algorithms application.

### BACKGROUND TO THE INVENTION

In the last 20 years, earthquakes have affected 125 million people, leading to around 750000 deaths. The threat from earthquakes disproportionately affects poorer countries, where a lack of building standards and ineffective response infrastructure often coexist. The most common cause of earthquake-related casualties is building collapse.

Search and rescue (SAR) is therefore an immediate priority. SAR may be defined as the location and extraction of trapped individuals, either informally by relatives and neighbors, or formally by professional local or international teams. Rescue may concern the removal of people from an exposed area either by professionals or other affected people. Rescue may only prevent loss of life if people are rescued before they will lose their life due to exposure.

The expected survival time for people in cold water is only a few hours, e.g., between 1 and 3 hours for a water temperature of 5° C. The floods with the greatest life loss have generally claimed their victims before professional rescuers were able to arrive. Rescue actions are expected to have a limited effect on fatalities in the direct impact phase, i.e., the first hours of the event.

Estimation of human vital signs is usually based on sensors with direct contact to the human body, e.g. a heart rate monitor, ECG etc. The breathing rate can also be estimated based on optical analysis by a video camera. To estimate heart rate and breathing rate of a human being from a distance, millimeter FMCW radars are available. However, their ranges are limited. Furthermore, walls, doors or other obstacles, even thick layers of clothes or heavy and thick duvets, can block their radar signals and prevent these systems from detecting or estimating human vital signs.

There is thus a need for a device and method for detecting people and their state in a more efficient, fast and reliable manner based on their vitals and behavior in situations where the existing sensors are not able to do so, most notably in a hazardous situation and through the obstacles with the goal to save people's lives. Additionally, the device/system and method may be used in non-SAR scenarios to detect vital signs of any given person in any suitable scenario such as, for example, in a hospital or in a car.

### SUMMARY OF THE INVENTION

The present invention accomplishes in particular accurate detection of presence of people and their physical state, based on one or more of their breathing rate, heart-rate, body position (whether stationary or moving, including motion rate and/or motion speed) and distance localization.

The invention is set out in the independent claims. Preferred embodiments of the invention are set out in the dependent claims.

We described a method for detecting a presence of one or more living objects by measuring one or more vital signs of the one or more living objects, the method comprising: transmitting, by a transceiver, a first signal; receiving, by the transceiver, a response to the first signal from any object in an environment from which at least a part of the transmitted first signal has been reflected off from the object towards the transceiver; translating the response into raw data; selecting a bin by processing and filtering the raw data based on a reflected energy received by the transceiver via the response, corresponding to a distance of a living object to the transceiver; sampling results from the bin obtained via the selection; analyzing the results to determine the presence and one or more vital signs of any living object from which the transmitted first signal has been reflected off towards the transceiver; outputting one or more vital signs parameters corresponding to the determined one or more vital signs.

Throughout the present disclosure, a living object may, for example, be a human or an animal.

The translating step, selecting step, sampling step, analyzing step and outputting step may be performed based on one or more algorithms (which run on a processor which may be coupled to the transceiver, for example via the processor and the transceiver both being comprised in the same device, or the processor/processing may run in a cloud computing environment).

The one or more algorithms may be complex algorithms which may be (intricate) sets of instructions designed for solving (intricate) computational problems which may necessitate multiple steps, advanced data structures and (sophisticated) logic. The one or more algorithms may hence be mathematical formulae applied in order to solve the computational problem(s).

The one or more vital signs parameters may be analyzed, which may allow for determining a trigger based on which an alert may be sent in case a vital risk is detected/determined.

In some examples, the method further comprises analyzing the one or more vital signs parameters to determine a physiological state of the one or more living objects.

In some examples, the method further comprises classifying a type of the one or more living objects via a combination of an amplitude of the reflected energy, an amplitude of an oscillating movement of the one or more living objects and the one or more vital signs. This may be performed via a shape of the determined received energy. In some examples, the type comprises a human and/or an animal. The classifying may be performed based on the one or more algorithms which runs on the processor.

In some examples, the method further comprises classifying an age of the one or more living objects by determining a size of the object via a combination of an amplitude of the reflected energy and the one or more vital signs.

In some examples, the signal is obtained from an ultra-wideband, UWB, device and/or from a plurality of UWB devices forming a distributed radar system.

In some examples, the one or more vital signs comprise one or more of a breathing rate, a heart-rate, a breathing rate variability and a heart-rate variability.

In some examples, the method further comprises combining the one or more vital signs parameters with one or more datasets obtained from one or more tertiary sensors (for example one or more video cameras and/or one or more biometrical sensors) to form a sensor fusion platform. The processor described herein on which the one or more algorithms run is configured to communicate with the one or more tertiary sensors. The one or more vital signs parameters together with the one or more datasets obtained from one or more tertiary sensors may thus be combined and analyzed, which may allow for determining a trigger based on which an alert may be sent in case a vital risk is detected/determined.

In some examples, the selecting comprises: calculating channel input responses, CIRs, based on the received response, wherein each of the CIRs is an output of the transceiver after the reflected first signal has been received by the transceiver; calculating the selected CIRs by applying a breathing extraction algorithm and/or a heart-rate extraction algorithm. The calculation of the CIRs and the calculation of the selected CIRs may be performed based on the one or more algorithms which runs on the processor.

In some examples, applying the breathing extraction algorithm and/or the heart-rate extraction algorithm comprises: calculating a covariance matrix of the subset of CIRs; calculating a complex sum of all columns of the covariance matrix to obtain a column array; calculating a power spectral density of each complex sum; finding a highest peak in the power spectral density; and analyzing the highest peak to determine the presence detection and one or more vital signs. The calculation steps and the finding step may be performed based on the one or more algorithms which runs on the processor.

In some examples, a preset number of CIRs per second is determined by the one or more algorithms.

In some examples, the part of the transmitted first signal is received by the transceiver due to propagation of the transmitted first signal.

In some examples, the received response is provided to a frequency converter configured to covert a frequency to a phase measured in a time domain.

In some examples, the method further comprises calculating a distance between the transceiver and the object based on a time of flight of the first signal, wherein the time of flight is a time between the transmission and reception of the first signal. The calculation may be performed based on the one or more algorithms which runs on the processor.

In some examples, the method further comprises sorting each CIR into a respective bin, wherein each bin represents a distance, or a range of distances, from the transceiver to the object. The sorting may be performed based on the one or more algorithms which runs on the processor.

In some examples, based on the finding of the peak in the power spectral density, the method comprises sending, to a display and/or alarm unit, a second signal comprising data indicating which bin the peak has been found in. The sending may be performed by the transceiver or processor and may be based on the one or more algorithms which runs on the processor.

In some examples, a covariance matrix is calculated as outlined further below by selecting a first period of time, whereby, preferably, the first period of time is 8 seconds or less.

In some examples, the method further comprises storing the covariance matrix in a memory, wherein each row of the matrix comprises a respective covariance.

In some examples, for calculating a covariance matrix, a second period of time is selected, whereby, preferably, the second period of time is 60 seconds or less.

In some examples, the subset of CIRs is 10% of the total number of CIRs.

In some examples, the method further comprises: checking if any of the peaks of the power spectral density calculation are harmonic frequencies, and finding a fundamental frequency. The checking and finding steps may be performed based on the one or more algorithms which runs on the processor.

In some examples, the highest peak of the power spectral density represents a vital signs parameter, in particular a respiration rate of a living object.

In some examples, the selecting comprises: sampling a preset number of channel input responses, CIRs, wherein the channel input response is an output of the transceiver after the reflected first signal has been received by the transceiver; sorting each CIR into a respective bin, wherein each bin represents a distance, or a range of distances, from the transceiver to the object; calculating a moving average of a time-dependent complex phasor of each bin using a filter (wherein the filter may be comprised in or coupled to the processor on which the one or more algorithms run); subtracting a current complex value of the filter from a current value of the time-dependent complex phasor; calculating a power spectral density of a complex phasor of a difference between the current complex value of the filter and the current value of the time-dependent complex phasor; and finding a peak in the power spectral density. The sampling, sorting, calculating, subtracting and finding steps may be performed based on the one or more algorithms which run on the processor.

In some examples, the method further comprises observing a subset of bins and perform the method from the calculation of the moving average step onwards using only the subset of bins. The observing step may be performed based on the one or more algorithms which run on the processor.

In some examples, the filter comprises a Kalman filter.

In some examples, the Kalman filter is configured to use a uniform acceleration motion model.

In some examples, the finding of the peak comprises finding the highest peak in a range of 30 to 180 Hertz.

In some examples, the method further comprises checking if any of the peaks of the power spectral density calculation are harmonic frequencies, and finding a fundamental frequency. The checking and finding steps may be performed based on the one or more algorithms which run on the processor.

In some examples, a peak of the power spectral density represents a vital signs parameter, in particular a heart rate of a living object.

In some examples, finding the peak comprises iteratively searching spectral lines on either side of a peak in a Fourier Transform of the power spectral density in a dichotomous manner.

In some examples, a Goertzel filter is used to find the peak.

In some examples, the number of CIRs is between 10 and 250 CIRs per second.

In some examples, based on the finding of the peak in the power spectral density, the method further comprises sending, to a display and/or alarm unit, a second signal comprising data indicating which bin the peak has been found in. The sending may be performed by the transceiver or processor and may be based on the one or more algorithms which run on the processor.

In some examples, only a subset of CIRs are sorted, and wherein the subset comprises 10% of the total number of CIRs.

In some examples, the part of the transmitted first signal is received by the transceiver due to propagation of the transmitted first signal.

In some examples, the method further comprises: obtaining, from one or more sensors, in particular one or more cameras and/or one or more biological sensors, data relative to the one or more living objects, and fusing the one or more vital signs parameters with the data obtained from the one or more sensors to predict a physiological state of the one or more living objects.

We further describe a device comprising: a transceiver configured to transmit a first signal and to receive a response to the first signal, wherein the response comprises at least a part of the transmitted first signal which has been reflected off of an object; and a computer-readable storage medium comprising instructions which, when executed by a processor, cause the processor to carry out the method of any one of the example implementations outlined above.

We further describe a method comprising: transmitting, by a transceiver, a first signal; receiving, by the transceiver, a response to the first signal, wherein the response comprises at least a part of the transmitted first signal which has been reflected off of an object; sampling, by a processor coupled to the transceiver, the received signal with a preset number of channel input responses, CIRs, wherein each of the channel input responses is an output of the transceiver after the reflected first signal has been received by the transceiver; optionally selecting a subset of the CIRs; calculating, by the processor, a covariance matrix of the subset of CIRs. The calculation of the covariance matrix preferably comprises, in some examples: a) selecting, by the processor, a first period of time located within a second period of time over which the response to the reflected first signal has been received, wherein the second period of time is longer than the first period of time; b) calculating, by the processor, a covariance of the first period of time to a rest of the longer period of time; c) moving, by the processor, the signal relating to the first period of time by one sample (i.e. shifting by one signal to re-align the correlation results in time) and calculating, by the processor, a covariance over the rest of the longer period of time; d) repeating, by the processor, steps a) to c) until the entire signal is used as part relating to the first period of time for the covariance calculation; e) collecting, by the processor, results obtained via steps a) to d) in a matrix in which every line values from different parts relating to the first period of time are stored, wherein every column of the matrix holds the covariance of the part relating to the first period of time and the corresponding signal; time aligning, by the processor, the matrix by circular shifting each line of the matrix by one sample; calculating, by the processor, a complex sum of all columns to obtain a column array; calculating, by the processor, a power spectral density of each complex sum (which may be done by applying a (Fast-) Fourier-Transformation following the calculation of the sum of each column); and finding, by the processor, a highest peak in the power spectral density. The frequency with the highest spectral density (i.e. with the highest peak in the power spectral density) may be considered the result (if in a range of about 7 to 50 breaths per minute or 30 to 200 beats per minute).

The first signal may be any suitable signal. In particular, the first signal may be an ultra-wideband, UWB, signal. In some examples, the transceiver may be able to transmit and/or receive UWB signals in a plurality of UWB bands. In some examples, the transceiver may be a transceiver which is commercially available. The transceiver may be able to receive UWB signals from an external device capable of transmitting UWB signals, wherein the received first signal mentioned above is transmitted from the external device. The UWB signals specified throughout the present disclosure may be UWB radio signals. Alternatively, any suitable signal may be used. In some examples, the method may be performed across multiple devices. That is to say, the first signal may be transmitted from a first device and the first signal may be received at a second device, wherein the remainder of the method may take place at least partially at the second device. In some examples, the first signal may be a pulsed radar signal.

In some cases, the transceiver may include 2, 3, or more antennas, for 1D, 2D, 3D and more radar detection and to achieve better resolution.

The processor may be part of an electronic circuit configured to transmit and/or receive UWB signals. Alternatively, the processor may receive the signal from the transceiver, while the processor is arranged remotely from the transceiver. The processing may, for example, be performed in the cloud.

Throughout the present disclosure, the object may be a person, an animal, a chair, a wall, or any other object. In particular, the object may be a chest of a person.

The covariance matrix and its associated steps may allow for a longer range of time to be analyzed and may therefore increase the chances of detecting a person, as opposed to a rogue signal being able to be signaled as a person. In some examples, this may lead to a respiration rate of the person being detected and estimated.

Whenever the term “object” is used throughout the present disclosure, this may be interchangeable with the term “surface”.

The above aspect may be used to detect respiration of a person and/or a respiration rate of a person.

The CIR may allow for the device to detect objects within the monitoring range of the device. The CIR may allow for the device to detect objects which are permanently within the monitoring range such as, for example, trees and flowers. The CIR may allow for the device to detect a distance of an object from the device and/or a size of an object. Throughout the present disclosure, a CIR may be defined as the answer (i.e. the channel output at the Rx) of the communication channel, given an UWB pulse as channel input (i.e. sent by Tx). The CIR may characterize the channel from Tx to Rx and is usually given as a function of time (often a “power-delay profile” is used, giving similar details of the channel). The time between transmission and receipt may be the “time of flight” and, hence, can be directly converted into a distance.

Although a person is mentioned throughout the disclosure, it is to be understood that the enclosed methods and devices may additionally or alternatively be configured to detect any living thing such as, for example, animals.

In some examples, only a part of the transmitted first signal is received by the transceiver due to propagation of the transmitted first signal and reflection of the signal in different directions by an object, so that only part of the reflected signal reaches the receiver/transceiver.

In some examples, the preset number of CIRs is as low as 25 and as high as 250 CIRs per second. In some examples, breathing rate estimation works with lower sampling rates than heart rate estimation. For breathing rate estimation, 25 CIRs/second may be used. The maximum rate (for example 250 CIRs/s) may be limited by the hardware. For heart rate estimation, more than 25 CIRs/s may be needed, for example 120 CIRs/s, but heart rate estimation may work well with higher rates as well. In some examples, there may be a minimum sampling rate that is needed for the procedures outlined herein and each vital sign.

In some examples, the processor is further configured to calculate a distance between the processor and the object based on a time of flight of the first signal, wherein the time of flight is a time between the transmission and reception of the first signal. This may allow for the processor to determine how far away the person is. This may allow for a person using a device which uses the method to better evaluate where the person is. This may particularly help in SAR scenarios.

In some examples, the processor is further configured to sort each received first signal into a respective bin, wherein each bin represents a distance, or a range of distances, from the transceiver to the object. This may allow for the processor to only sample CIRs and/or calculate the covariance matrix of certain distances. The bins to be used may be automatically decided by the processor based on which bins comprise received signals that are most promising for the indication of a person and/or the bins to be used may be manually selected and/or the bins to be used may be preset. The automatic decision may be aided and/or guided by the use of a machine learning module couplable to the processor. This may allow for the user to better determine how far the person is away from the device using the method. In some examples, the values of the bins may be customizable by a user and/or preset. This may be particularly advantageous in SAR scenarios. This may also reduce the processing power needed to be used by the processor, as only a subset of the received first signals are used for the remainder of the method.

In some examples, based on the finding of the peak in the power spectral density, the processor is configured to send, to a display and/or alarm unit couplable to the processor, a second signal comprising data indicating which bin the peak has been found in. This may allow for the user to better identify where a person may be.

In some examples, the first period of time is 8 seconds. A length of 8s works well for estimating the breathing rate. It may, in some examples, be too long for estimating the heart rate with a similar procedure. The length of the segment used for the correlation may depend on the period duration of the measured oscillation. The length of the segment (i.e. period) may be varied to find a suitable length.

In some examples, the results obtained via the processor may be stored in a memory for post-processing and analysis (in particular offline processing).

In some examples, the second period of time is 60 seconds. A length of 60s works well for estimating the breathing rate. The length of the segment used for the correlation may depend on the period duration of the measured oscillation. The length of the segment (i.e. period) may be varied to find a suitable length.

In some examples, the calculation of the complex sum results in a column array.

In some examples, the power spectral density is calculated based on the column array.

In some examples, the preset number of CIRs are not sampled in real time. This may allow for the readings to be taken, and then analyzed later. This may allow for the method and/or processor and/or device to be further trained to recognize what signals reflected from a person look like. Resultantly, this may improve the accuracy of the method. In some examples, this offline processing may be used to train a machine learning module, to which the processor may be couplable to. The machine learning module may be used to improve the detection of a person should the CIRs be sampled in real time.

In some examples, the preset number of CIRs are sampled via batch processing.

In some examples, the preset number of CIRs are sampled in real time. This may be used in SAR scenarios to detect a person in real time, thereby aiding the user of the device which uses the method in finding people.

In some examples, the subset of CIRs is 10% of the preset number of CIRs. This may allow for a reduction in processing power, while still achieving an acceptable result. Any percentage of the preset CIRs may be used, such as, for example, 15%, 20%, 25%, 30%, 35%, 40%, 45%, 50%, 55%, 60%, 65%, 70%, 75%, 80%, 85%, 90% or 95%.

In some examples, the method further comprises checking, by the processor, if any of the peaks of the power spectral density calculation are harmonic frequencies. If there are harmonics, then one may use the first harmonic (the fundamental frequency) as the relevant (e.g. breathing or heart) rate, even if at this frequency there is not the highest peak. In some examples, the method further comprises finding, by the processor, a fundamental frequency. This may allow for the peak to be found.

In some examples, a highest peak of the power spectral density represents a respiration or heart rate of a person. The processor may then indicate to the user the vital signs of the person.

We further describe a method comprising: transmitting, by a transceiver, a first signal; receiving, by the transceiver, a response to the first signal, wherein the response comprises at least a part of the transmitted first signal which has been reflected off of an object; sampling, by a processor coupled to the transceiver, a preset number of channel input responses, CIRs, wherein the channel input response is an output of the transceiver after the reflected first signal has been received by the transceiver; sorting, by the processor, each CIR into a respective bin, wherein each bin represents a distance, or a range of distances, from the transceiver to the object; calculating, by the processor, a moving average of a time-dependent complex phasor of each bin using a filter, wherein the filter is comprised in or coupled to the processor; subtracting, by the processor, a current complex value of the filter from a current value of the time-dependent complex phasor (whereby the current complex value of the filter is the output of the moving average filter); calculating, by the processor, a power spectral density of a complex phasor of a difference between the current complex value of the filter and the current value of the time-dependent complex phasor; and finding, by the processor, a peak in the power spectral density. Due to multi-path interference, there may be a sum of different (partly time-dependent) complex phasors adding up to the time-dependent complex phasor one measures at the Rx antenna. The time-dependent mean of this complex phasor may be compensated by subtracting the output of a moving average filter (applied to the phasor) and the phasor itself. The resulting complex phasor, preferably, contains only the oscillation due to breathing/heart beats. An FFT (Fast-Fourier Transformation) is used to calculate the power spectral density of the remaining (difference) signal. The frequency with the highest power spectral density is found (the highest peak) and this is the frequency estimation of the vital sign of interest.

The first signal may be any suitable signal. In particular, the first signal may be an ultra-wideband, UWB, signal. In some examples, the transceiver may be able to transmit and/or receive UWB signals in a plurality of UWB bands. In some examples, the transceiver may be a transceiver which is commercially available. The transceiver may be able to receive UWB signals from an external device capable of transmitting UWB signals, wherein the received first signal mentioned above is transmitted from the external device. The UWB signals specified throughout the present disclosure may be UWB radio signals. Alternatively, any suitable signal may be used. In some examples, the method may be performed across multiple devices. That is to say, the first signal may be transmitted from a first device and the first signal may be received at a second device, wherein the remainder of the method may take place at least partially at the second device.

Throughout the present disclosure, the processor may be part of an electronic circuit (printed circuit board, PCB) configured to transmit and/or receive UWB signals. In some examples, however, the processor may be remote from the transceiver, e.g. processing may take place in a cloud computing environment.

The subtraction may allow for the calculation of the power spectral density to be easier, thereby reducing the processing power needed in the method (as outlined above).

The finding of a peak of the power spectral density may allow for signals reflected from a stationary surface to be discarded, as these signals may not have a peak, but may be substantially constant within the power spectral density calculation.

The above-described aspect/method may be used to detect a heart rate of a person and/or a heart rate variability of a person.

In some examples, the processor is configured to observe a subset of bins, and perform the method from the calculation of the moving average step onwards using only the subset of bins. This may mean that the remainder of the method applies only to the observed bins. This may allow for the processor to only sample and/or calculate the covariance matrix and/or sample the CIRs of certain distances. The bins to be used may be automatically decided by the processor based on which bins comprise received signals that are most promising in relation to the detection of a person and/or the bins to be used may be manually selected and/or the bins to be used may be preset. The automatic decision may be aided and/or guided by the use of a machine learning module couplable to the processor. This may allow for the user to better determine how far the person is away from the device using the method. In some examples, the values of the bins may be customizable by a user and/or preset. This may be particularly advantageous in SAR scenarios. This may also reduce the processing power needed to be used by the processor, as only a subset of the received first signals are used for the remainder of the method.

In some examples, the filter is a Kalman filter. This may allow for an “off the shelf” part to be used in the processor, thereby reducing the cost of the processor and therefore, the device using the method. Other types of filters are possible.

In some examples, the Kalman filter is configured to use a uniform acceleration motion model. A Kalman filter may, in some examples, need a model to be used in parallel to the observed measurements. It is a filter applying a “prediction-correction” operation. An important advantage is that the Kalman filter is not introducing a delay; a standard moving average filter could also be used, however they do not include a motion model and they would add a delay.

In some examples, the calculation of the power spectral density comprises using a result of the subtraction. This may allow for the calculation of the power spectral density to be easier, thereby reducing the processing power needed in the method. The Kalman filter may be used to subtract the time-dependent mean of the complex phasor (as a kind of moving average filter).

In some examples, the finding of the peak comprises finding the highest peak in a range of 30 to 180 Hertz. This may allow for the method to discard any readings which may not be a heart rate and/or heartbeat, thereby improving the accuracy of finding a person. The algorithm may, in some examples, only accept frequencies in an a priori given range. The maximum power spectral density is determined only in this range. The range of 30 Hz to 180 Hz may, in some examples, make only sense in case of estimating the heart rate.

In some examples, the method further comprises checking, by the processor, if any of the peaks of the power spectral density calculation are harmonic frequencies (as outlined above).

In some examples, the method further comprises finding, by the processor, a fundamental frequency (as outlined above). This may allow for the peak to be found.

In some examples, a highest peak of the power spectral density represents a heart rate of a person. The processor may then indicate to the user the vital signs of the person.

In some examples, finding the peak comprises iteratively searching spectral lines on either side of a peak in a Fourier Transform of the power spectral density in a dichotomous manner. This may allow for a heartbeat of a person to be detected with greater accuracy.

In some examples, a Goertzel filter is used to find the peak. This filter may be used to find the peak. A Goertzel filter (Goertzel algorithm) may be particularly advantageous as a higher resolution in the frequency domain may be achieved.

In some examples, the number of preset CIRs is between 25 and 250 CIRs per second. These sampling rates may be used for heart rate estimation. Lower rates (below a certain value) may not allow a precise estimation. For breathing rate estimation, lower rates may be used, too. The reason is that the breathing frequency is usually lower than the heart rate.

In some examples, based on the finding of the peak in the power spectral density, the processor is configured to send, to a display and/or alarm unit couplable to the processor, a second signal comprising data indicating which bin the peak has been found in. This may allow for the user to better identify where a person may be.

In some examples, the preset number of CIRs are not sampled in real time. This may allow for the readings to be taken, and then analyzed later. This may allow for the method and/or processor and/or device to be further trained to recognize what signals reflected from a person look like. Resultantly, this may improve the accuracy of the method. In some examples, this offline processing may be used to train a machine learning module, to which the processor may be couplable to. The machine learning module may be used to improve the detection of a person should the CIRs be sampled in real time.

In some examples, the preset number of CIRs are sampled via batch processing. In some examples, the offline mode may be more precise and accurate than the online mode.

In some examples, the preset number of CIRs are sampled in real time. This may be used in SAR scenarios to detect a person in real time, thereby aiding the user of the device which uses the method in finding people.

In some examples, only a part of the transmitted first signal is received by the transceiver due to reflection and propagation of the signal in different directions such that only a part of the transmitted signal is received at the transceiver.

In some examples, the subset of CIRs is 10% of the preset number of CIRs. This may allow for a reduction in processing power, while still achieving an acceptable result. Any percentage of the preset CIRs may be used, such as, for example, 15%, 20%, 25%, 30%, 35%, 40%, 45%, 50%, 55%, 60%, 65%, 70%, 75%, 80%, 85%, 90% or 95%.

We further describe a device comprising: a transceiver configured to transmit a first signal and to receive a response to the first signal, wherein the response comprises at least a part of the transmitted first signal which has been reflected off of an object; and a computer-readable storage medium comprising instructions which, when executed by a processor, cause the processor to carry out the steps of any one or more of the methods outlined above.

Even if some of the features described above have been described in reference to a particular method, these features may also be in reference to another method and vice versa. These aspects may also apply to a device and/or system for detecting a person.

For the methods described herein, said methods may comprise only some of the steps mentioned herein. In some examples, some steps may be performed sequentially and/or some steps may be performed concurrently. In some examples, some of the steps may take place in a different order to those described herein, as long as the result is the same.

### BRIEF DESCRIPTION OF THE DRAWINGS

These and other aspects of the invention will now be further described, by way of example only, with reference to the accompanying figures, wherein like reference numerals refer to like parts, and in which:

FIGS. 1a and 1b show a system for detecting a person according to some examples as described herein;

FIG. 2 shows a block diagram of a method according to some examples as described herein;

FIG. 3 shows a depiction of a time-variable phasor according to some examples as described herein;

FIG. 4 shows a system for detecting body parts of a person for body movement according to some examples as described herein.

FIG. 5 shows time-variable phasor according to some examples as described herein;

FIG. 6 shows a flow diagram of a heart-rate calculation according to some examples as described herein;

FIG. 7 shows a flow diagram of a heart-rate extraction algorithm according to some examples as described herein;

FIG. 8 shows a flow diagram of a breathing rate extraction algorithm according to some examples as described herein;

FIG. 9 shows a flow diagram of a method for detecting a person according to some examples as described herein;

FIG. 10 shows a flow diagram of a method for detecting a person according to some examples as described herein;

FIG. 11 shows a schematic illustration of a device for detecting a person according to some examples as described herein;

FIG. 12 shows a system for detecting a person according to some examples as described herein;

FIG. 13 shows a superimposition of breathing and heart signals time-variable phasor according to some examples as described herein;

FIG. 14 shows a superimposition of breathing and heart signals time-variable phasor according to some examples as described herein;

FIG. 15 shows a received signal according to some examples as described herein;

FIG. 16 shows a graph of a comparison of heart rate measurements according to some examples as described herein;

FIG. 17 shows a result of a method according to some examples as described herein; and

FIG. 18 shows graphs of results from a method according to some examples as described herein.

### DETAILED DESCRIPTION OF THE INVENTION

The methods, devices and systems described herein can be used on the inside or outside of premises and/or buildings, in, for example, healthcare and/or search and rescue missions and/or inside of moving objects such as vehicles. The methods, devices and systems described herein can be used to detect a single person, or a group of persons, based on their movements and/or their radar cross sections and/or their vital signs. When there is a distance between people of more than 30 centimeters, their radar echoes may be in different bins. Each bin contains then only the signal from one person. Therefore, people can be counted and their vital signs can be detected, as is done for a given bin.

The range for detecting human beings may be as close as 50 centimeters and up to 50 meters in open field, and the detection range of vital signs may be reduced to half of this value. To reliably estimate the heart rate of a living object, the range may be further reduced to 3 meters, while estimating the breathing rate is possible for larger distances due to the increased body movement in case of breathing. The detection range limitation may be managed through the methods described herein.

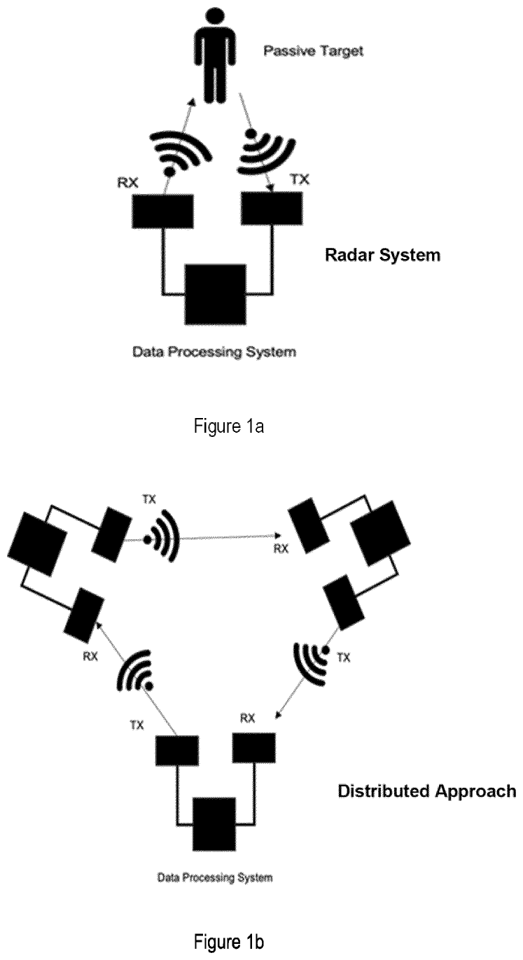

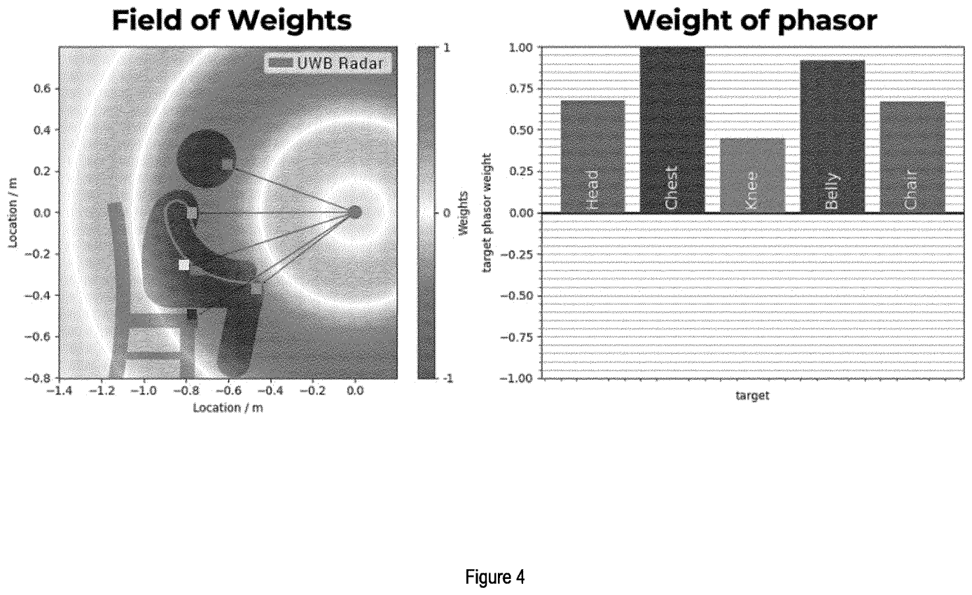

To detect the presence of vital signs of people, the methods may allow for the monitoring of the involved small movements of the bodies, e.g., the chest movements due to breathing and due to the changes of the blood flow in the large blood vessels (see FIG. 4). Amplitude, as well as phase measurements of the time domain UWB radar signal in the complex baseband, may be used, combined with a specific signal processing based on windowing, filtering, and spectral analysis. Radar may simply mean that there is a transmitter and receiver, one transmits and one receives a signal. The radar signal typically comes from the same device (i.e. Tx antenna and Rx antenna are on the same device and they are synchronized in time). Further, The same device might not be needed, as one can use Rx and Tx from multiple devices operating on the same frequency via creating a ‘mesh’ or ‘virtual’ (distributed) radar system (see FIG. 1b). As an example, one Rx from one device captures this signal from the second device (Tx) that carries, e.g., Ipatov sequences, and synch it in the time domain. Having a ‘virtual’ radar configuration allows to optimize the usage of devices and allows to simply use, e.g., the UWB communicating protocol IEE 802.15.4z without having a ‘radar’ chip or ‘radar’ single device. All methods described throughout the present disclosure may not be only limited to the ‘radar’ signal, but can be used by any device that has a UWB chip. As an example, a UWB chip is installed in smartphones, smartwatches, and, in almost every car for keyless access.

In some examples, for each estimate of CIR 1024 Ipatov sequences are used. Using a preamble consisting of several Ipatov sequences, the receiver chip measures the communication channel before receiving a packet and generates a CIR. In this sense, the CIR is a by-product which serves to improve the communication quality. When the transmitter and receiver chips are put into operation next to each other and a direct path between the systems is prevented, communication takes place mainly via reflections in the environment. The generated CIR then has the same interpretation as for a radar system.

To estimate the vital signs of people, the methods may allow for the monitoring of the involved small oscillating movements of the body, e.g., the periodic chest movements due to breathing and due to the periodic changes of the blood flow in the large blood vessels. Additional movements of the body may make the estimation much more difficult, as they usually show larger movement amplitudes. In some examples, the methods outlined herein may try to account for these problems, either by the covariance method or by the Kalman filter, or a combination of both. A first step could be to omit all measured CIRs which contain very strong movements (outlier detection), as the methods may not be able to cope with all measures in case the amplitudes are too large. Phase measurements of the time domain UWB radar signal in the complex baseband may be used for the estimation of vital signs, and may be combined with a specific signal processing based on windowing, specific filtering, outlier detection and exclusion, spectral analysis, breathing event detection, and heart beat detection. This may not be based on phase measurements only, but may contain the amplitude/complex signal (given by magnitude and phase). Covariance/correlation methods may also be applied, as well as outlier detection.

To detect living beings that may be buried or trapped, a UWB radar signal may be able to penetrate the blocking materials. These materials may have different variables such as, for example, thickness and type of structure (wood, stone, glass), which may result in weaker signals, reflection and propagation. The problem of the signal penetration through various surfaces may be addressed through the [A] circuitry/PCB design and specific optimized antenna design as well as [B] signal measuring algorithms related thereto-including windowing, specific filtering, spectral analysis, outliers' exclusion, anomalies detection, and treatment in a given timeframe.

UWB technology, which may be used herein, uses very broadband signals (at least 500 MHz, up to several GHz bandwidth), with center frequencies between 3.5 GHz and 8 GHz. At least one suitable UWB communication standard is available: IEEE 802.15.4z. This standard and/or UWB signals in general may be used in the methods, devices and systems mentioned herein.

The methods, devices and systems mentioned herein may be based on a measurement of a channel impulse response (CIR) between a transmitter and a receiver. A UWB CIR may be defined as the answer (i.e. the channel output at the Rx and/or receiver) of the communication channel, given a UWB pulse as channel input (i.e. sent by Tx and/or transmitter). This may characterize the channel from Tx to Rx, and may be given as a function of time (often a “power-delay profile” is used, giving similar details of the channel). The time may be the “time of flight” and, hence, this can be directly converted into a distance by the processor described herein.

The UWB CIR may allow for insights and conclusions about the environment. For instance, in the case there is a direct (unobstructed) path (line-of-sight—LOS) between Tx and Rx with a distance x0, for the signal it takes to =x0/c to travel from Tx to Rx (c being the velocity of the electromagnetic wave, in vacuum the speed of light). Hence, the UWB CIR may show a corresponding amplitude at time to.

Particularly, reflections of the transmit signal may be visible in a CIR. This may be considered as an example of a reflection leading to a path between Tx and Rx of length xi (xi>x0)—corresponding to a path duration of t1 (with t1>t0, i.e. this path is delayed compared to the LOS).

Hence, based on UWB CIR reflectors/scatterers, i.e. structures that reflect/scatter the UWB signal back, can be identified, and the corresponding time of flight (or path length) can be determined. Due to the large bandwidth of the UWB signal used, the temporal (and therefore also the spatial) resolution of the CIR may be very high.

A UWB CIR may be described in the equivalent (or complex) baseband. The “taps” (isolated and small amplitudes in the CIR, typically from LOS or a single strong reflector) or the clusters (of several reflectors or scatterers leading to a spread of the corresponding amplitude(s)) in the CIR, may be complex values, displayed by magnitude and phase (or a real and an imaginary part).

A surface, i.e. the chest or the throat of the person in front of the radar, may reflect the radar signal. This surface may move according to the breathing or the pulse of the person. Therefore, there may be (small) changes in the distance between UWB radar and surface. The methods, devices and system mentioned herein may use these variations in distance to estimate breathing and/or heart rate.

The first step may be to identify that part of the CIR that is showing the reflection of the surface. This “bin” of the CIR—which may correspond to a distance of the person under test to the Rx antenna and/or receiver—may be extracted from the CIR results. The bin may be a complex number (magnitude and phase). In principle, the phase signal, observed over time, may allow for the measurement of very small distances (or changes in distances) with high precision. The phase values—observed between consecutive measurements—may define a time-dependent complex phasor, which can be used to estimate a frequency. It is to be noted that not only the phase values, but also the complex values—including phase and magnitude—may be used. The phase values only may, in some scenarios, be suboptimal, however they offer a low-complexity solution (and in other context/scenarios they are all that may be needed). From the phases φ(t) (given as phase values sampled at specific time instants) we define complex values by taking them as exponent of complex e-functions (e∧(jφ(t)).

For a given time index, the complex value of the considered bin of the UWB CIR may be the sum of some or all reflections arriving at the Rx antenna and/or receiver from the same distance. In some examples, this may be from distances of approximately +/−10 to 15 cm from the center of the bin. The skilled person understands that this value may change depending on the scenario and/or capabilities of the method and/or device and/or system. For the estimation, a higher bandwidth may, in some examples, be better, leading to a smaller interval measured from the center of the bin. Preferably, there is only one reflection in the bin, because then there is no problem with interfering signals.

The surface detected by the methods, devices and systems mentioned herein, i.e. the chest or throat, may be only one source of such a reflection, with others being, for example, a chair the person is using, or a non-moving bone inside the body of the person, or other suitable object. This may mean that several complex numbers (one per “significant” reflector/scatterer) may sum up to the complex value of the bin. Therefore, it may not be straight forward to identify the time-dependent complex phasor of the surface the method/device/system is attempting to analyze.

Two approaches may be used to estimate the respiration rate and/or heart rate of the person. The first one may be specifically designed for “offline” processing, i.e., the intention may not be to monitor the vital signs in real-time, but rather to take a high number of CIRs into account to allow for reliable estimates.

The second approach may be designed for “online” processing, i.e. it can be used to monitor vital signs in real-time. Both approaches may assume that only breathing, or the pulse, is causing movements included in the complex phasor of the considered bin, whereas the other reflectors are assumed to be static. The skilled person understands that this does not hold for all situations, e.g., when the person is moving. Hence, the methods described herein may collect the measured CIRs which do not show very strong movements, as these are typically not due to breathing or pulse. In such a scenario, the method may discard a bin where there are no, or few, CIRs which show movement. For a given bin only values from such CIRs may be used that show in this specific bin no large movements, i.e. large amplitudes of the complex phasor. This could be an outlier detection based on threshold detection.

The embodiments described herein may relate to non-intrusive wireless systems and methods configured to detect the presence of living beings and estimate their vital signs—even through obstacles. Said embodiments can be used for non-intrusive people monitoring in security solutions, in healthcare, and also for detecting buried or trapped living people after hazardous events, such as earthquakes, landslides, floods etc. The skilled person understands that the embodiments mentioned herein are not limited to the detection and estimation of the vital signs of human beings, but can also be used for animals of a certain size, e.g. pets or cattle.

In addition, the methods, devices and systems mentioned herein may also be achieved in conjunction with gathering and fusing data from other “sensing” devices such as, for example, cameras, sensors, or the like. The methods, devices and systems described herein may form part of a sensor fusion platform relating to human vitals and biological signatures. The resulting model of such a platform may comprise collecting and sending data, and processing said data by local embedded processing nodes, which may be more accurate for vitals detection and building predictive models around human behavioral patterns as the model may balance the strengths of different sensors.

As mentioned herein, the methods and/or devices may be configured to measure one or more of the breathing rate and the heart-rate and the heart-rate variability (also referred to as vital signs) of a person by using a radar based on the UWB technology. During the measurement, the channel impulse response may be estimated at a rate of 25 CIRs per second. When observing a time varying signal such as breathing or heart-rate, multiple CIRs may need to be considered. It may also be important that the time interval τ_{CIR} between two CIR estimations is constant since it may be equivalent to a sample rate which may play a major role in the signal processing chain. Typically, a person corresponds to a tap in the CIR, slight movements of the body may be reflected in the CIR as phase and magnitude changes of the taps. Observing those changes over a given period makes it possible to extract the periodic behavior of a person's body movements on a small scale.

The measurement system may be split into two parts: the sensor, and the processing unit part. The sensor may be composed of a Printed Circuit Board, PCB, featuring one UWB chip, or any other number of chips and/or suitable type of chips, at least one transmitter, at least one receiver and a microcontroller unit, MCU. The skilled person understands that there may be fewer, or more parts to the radar than those mentioned herein. Two antennas may be attached to the radar PCB via SMA cables, or any other suitable type of cable. The radar may continuously perform CIR estimations and push the data to the processing unit.

The data processing may take place on the processing unit, where the data may be parsed and run through the vital signs detection algorithm. The output of the processing unit algorithms may consist of an estimation of the breathing and/or heart-rate. Such a system is shown in FIGS. 1a and 1b.

It is clear to a person skilled in the art that the statements set forth herein may be implemented under use of hardware circuits, (complex) algorithms means or a combination thereof. The (complex) algorithms means can be related to programmed microprocessors or a general computer, an ASIC (Application Specific Integrated Circuit) and/or DSPs (Digital Signal Processors). For example, the processing unit may be implemented at least partially as a computer, a logical circuit, an FPGA (Field Programmable Gate Array), a processor (for example, a microprocessor, microcontroller (μC) or an array processor)/a core/a CPU (Central Processing Unit), an FPU (Floating Point Unit), a ECU (Electronic Control Unit), a VCU (Vehicle Control Unit), a NPU (Numeric Processing Unit), an ALU (Arithmetic Logical Unit), a Coprocessor (further microprocessor for supporting a main processor (CPU)), a GPGPU (General Purpose Computation on Graphics Processing Unit), a multi-core processor (for parallel computing, such as simultaneously performing arithmetic operations on multiple main processor(s) and/or graphical processor(s)) or a DSP.

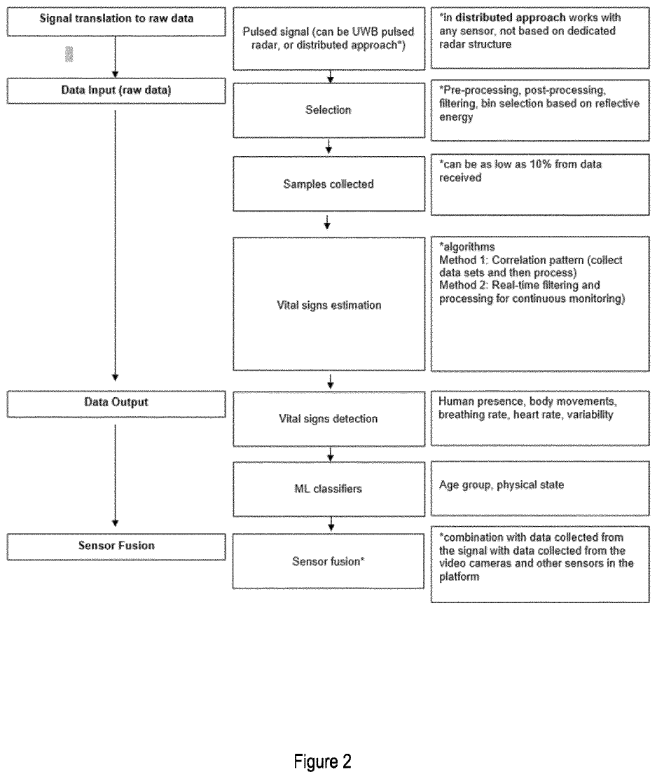

FIG. 2 shows a block diagram of a method of detection according to some examples as described herein.

As can be seen, a pulsed signal, such as, for example, a UWB pulsed radar or a distributed approach where multiple antenna and/or transceivers and/or receivers and/or sensors are used to translate the signal into raw data.

A bin is then selected, based on the reflected signal, wherein the bin(s) represent data where a person could be located. A set of samples may then be collected from the received signals, wherein 10% or more of the received signals are sampled. The subset of signals sampled may be any randomly chosen subset of signals from the received signals. Following this, a correlation pattern, as mentioned herein, can be used and/or a Kalman filter, as mentioned herein, can be used. This may allow for an improvement in accuracy of determining the breathing rate and/or the heart-rate of a person and/or in the case of real time monitoring, this may allow for a faster determination of these parameters by up to 20 seconds.

Then, the breathing rate and/or heart-rate can be determined via the methods mentioned herein and/or machine learning can be used as described herein.

Additionally, the data from the sensors and/or the output from the method may be fused with further data in order to better determine the condition and/or vital signs of a person.

The firmware running on the radar system may send UWB packets, and may receive packets in an endless loop with a rate of a predetermined number of packets per second. The predetermine number of packets may, for example, be 200 (corresponding to the 200 CIRs per second mentioned above). It may, however, in some examples be any number between 10 and 250 CIRs per second. This number is defined (e.g. by an engineer) and it is stored in the config file. The lower the number of CIRs, the lower the power consumption of the chip. However, with such a low number of packets (or CIRs) or samples of the raw data/inputs, it may be more challenging to detect breathing, as 25 CIRs may be too small a sample. Thus, it may take time and may reduce accuracy. However, with the methods described herein, breathing with such as short rate can be detected within less than 10 seconds and with 95% accuracy, which is important for rapid response.

From each packet exchange, a CIR can be extracted. The radar system may only gather the raw data of the CIR, and send them to the Data Processing System. The radar system may operate at a center frequency f_{c} of 6.5 GHz with a Bandwidth B of 500 MHz, which may correspond to a relative Bandwidth B_{f} of 7.69%.

$${B}_{f} = \frac{B}{{f}_{c}}$$

This may satisfy a condition for the signal to be considered as UWB, namely that the used pulse has a bandwidth of 500 MHz, but not a further condition which states that the relative bandwidth of the system should be at least 20%. The pulse bandwidth may be a limiting factor for detecting targets that are near to each other.

The approach described herein may maximize the chances of observing vital signs with the radar system. This may lead to a set of assumptions and constraints that aim to provide a best scenario environment for the measurement, for which at least one may be fulfilled:
1. The target person may be located in the antenna boresight, that is, the axis of maximum gain;
2. The vital signs observation may happen only on one person;
3. There may be no parasitic movements in the neighboring bins, ideally over the whole distance range;
4. The target person may sit on a chair and lean back against the chair to avoid swaying movements; and
5. The target person may not move their head or arms during the measurement, the only moving part may be the chest, due to respiration.

The skilled person understands that not all of the above conditions may be fulfilled. In this case, the detection of multiple people and/or of a moving person is still possible, but it may be more difficult.

For a single moving target within one bin, the complex channel impulse response may be used to estimate the vital signs. In this way, each bin can be modeled as a complex phasor s(t) with time-varying amplitude and phase.

$$s ( t ) = a ( t ) \cdot {e}^{j ϕ ( t )}$$

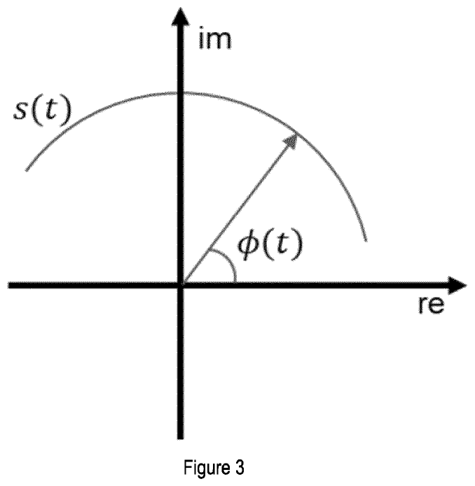

A representation of the above is shown in FIG. 3.

For multiple moving targets within one bin, or multiple bins, multiple overlapped signals can be modeled as the sum of amplitude modulated phasors with a time varying phase.

$${S}_{tot} ( t ) = {\sum }_{i = 0}^{n} {a}_{i} ( t ) \cdot {e}^{j {ϕ}_{i} ( t )}$$

In the initial consideration, only one phasor may have a time varying phase, and it is assumed that there are no amplitude fluctuations. With any number of static targets and one time-varying phasor, the trace of the signal may correspond to an arc with its center offset form the origin.

For moving objects in each bin, the amplitude variation may be due to changes in a distance of the target form the receiver and/or transmitter and/or transceiver and/or device, and therefore path loss variations, as well as changes in reflectivity of the object to account for different orientations of the reflector with respect to the radar, may be represented. Another reason for the amplitude variability within a bin may be tied to the shape of the Radio Resource Control, RRC, pulse and the fact that it may not be a perfect rectangle. Thus, depending on the position in the bin, the amplitude of the target may be scaled with the corresponding value of the RRC pulse.

Small distance variations may also influence the phase of the carrier signal leading to a variation of the phase in the CIR.

This may make it clear that a target moving within a bin can be seen as a complex phasor with changing amplitude and phase.

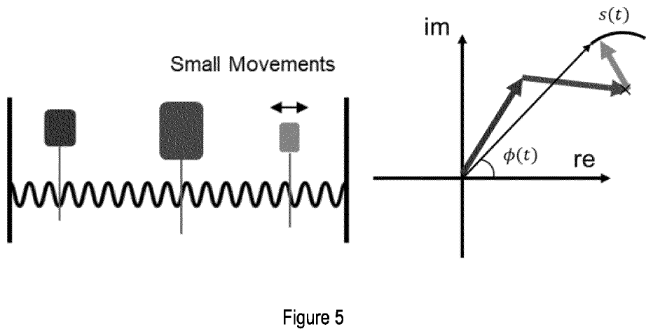

In the case of multiple targets located in the same bin, the observed amplitude and phase of that bin may be the sum of each target, which can be seen as a vector addition. Typically, there are multiple moving targets in the same bin, which renders the amplitude and phase of the measured result also time variable. Furthermore, the individual components contributing to the measured result may not be able to be resolved since the receiving antenna may only observe the sum of all signals. This is shown in FIGS. 4 and 5.

The motion model may uniformly accelerate, although any suitable model can be used, which can be expressed by the equation:

$$\frac{1}{2} {at}^{2} + {v}_{0} t + {s}_{0}$$

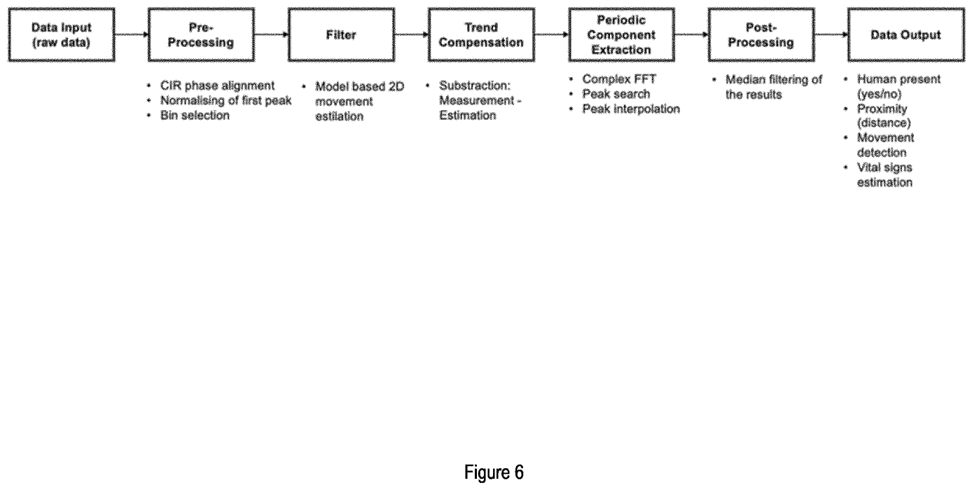

If the acceleration was left constant, the motion may happen in a straight line. Therefore, the acceleration may be driven by the process noise of the Kalman filter. By tweaking the process noise, one can determine how closely the filtered signal follows the original signal. This may allow for the motion generated through breathing to be followed, but not the motion induced by the heartbeat. An overview of the heart-rate calculation is shown in FIG. 6.

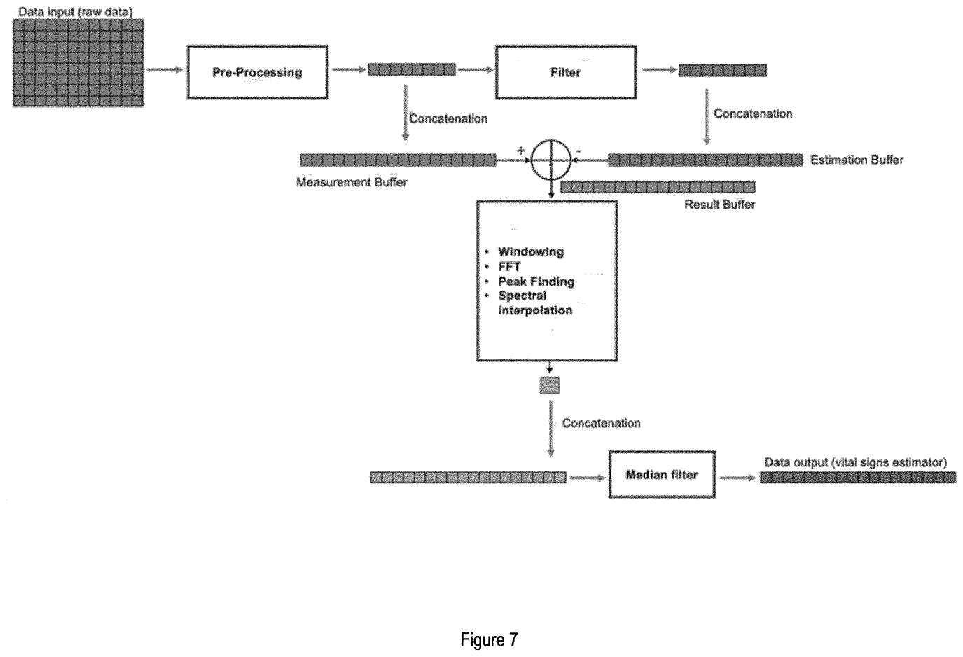

The FFT may be computed on the complex data, and the peak search may be done based on prominence and minimal amplitude criteria. Individual spectral lines may be computed based on a Goertzel filter to provide a finer frequency resolution, although any other suitable filter/filtering technique can be used. The true peak may be searched for in an iterative manner in a dichotomous way by calculating spectral lines left and right of the peak in the FFT. A more detailed view of the heart-rate extraction algorithm is shown in FIG. 7.

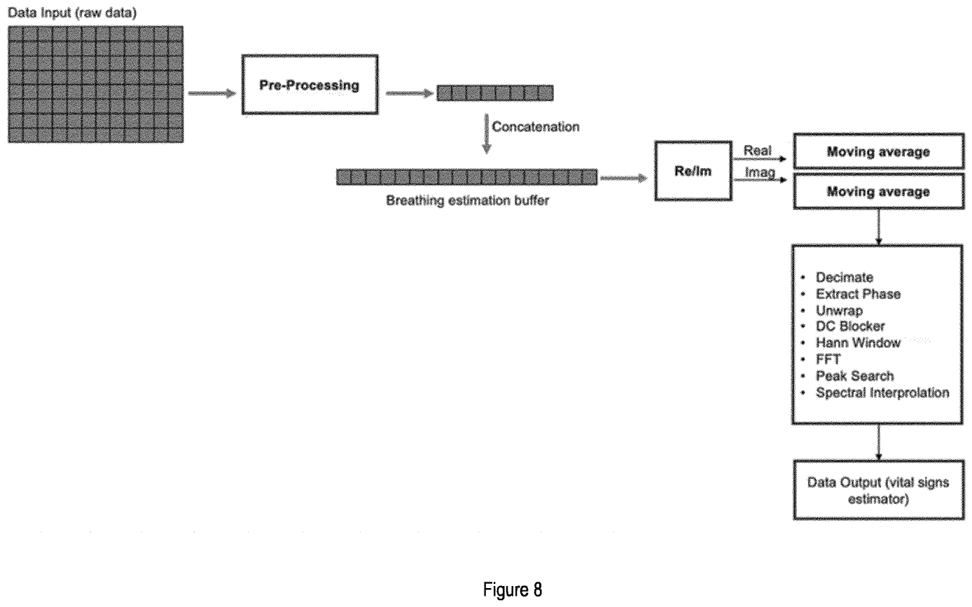

The breathing rate extraction may be simpler than the heart-rate extraction, since it is the major signal that may be observed, as seen in FIG. 15. In this way, the breathing rate may be extracted by observing the phase of the raw signal. As mentioned above, the signal generated by the breathing may rotate around a point in the complex plane. This rotation may induce a phase change that is periodic, even if the signal is not centered around the origin. The benefit of this method is its simplicity because it may disregard the complexity of static targets offsetting the center of rotation. This is shown in FIG. 8.

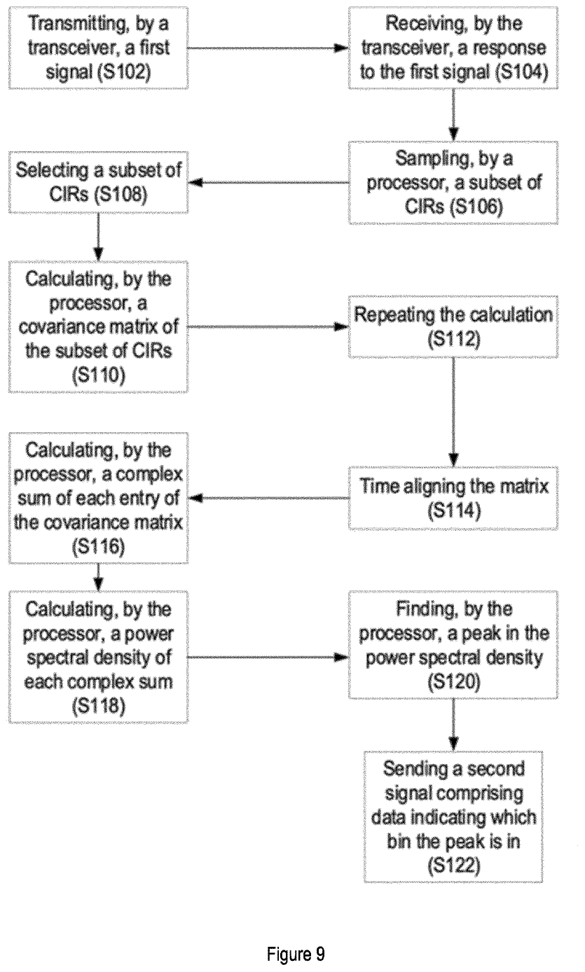

FIG. 9 shows a flow diagram of a method for detecting a person according to some examples as described herein.

In the method of FIG. 9, the method begins by transmitting, by a transceiver, a first signal S102. The first signal may be a UWB signal as described herein. In some examples, a transmitter may be used instead of a transceiver. A response to the first signal may then be received by the transceiver S104. The response may comprise at least a part of the transmitted first signal which has been reflected off of a surface. The surface may be a person, as described herein. In some examples, a receiver may be used instead of a transceiver.

The processor, in some examples, may then calculate a distance between the processor and the surface based on a time of flight of the first signal, wherein the time of flight is a time between the transmission and reception of the first signal. This may result in the processor calculating a distance to the person.

The processor, in some examples, may then sort each received first signal into a respective bin, wherein each bin represents a distance, or a range of distances, from the transceiver to the surface. Examples of bins may be, for example, 1 to 1.99 meters, 2 to 2.99 meters, or any other suitable distance or range. In some examples, the bin may be automatically set by a user of a device which uses the method and/or be a preset bin.

In some examples, the method may comprise sampling by a processor couplable to the transceiver, the received signal with a preset number of channel input responses, CIRs, wherein each of the channel input responses is an output of the transceiver after the reflected first signal has been received S106. Here, for example, only 10% of the received signals and/or CIRs may be used for the remainder of the method. The signals and/or CIRs used may be randomly chosen, or every 10th signal and/or CIR.

In some examples, the method may further comprise selecting a subset of the CIRs S108. This may be done in conjunction with at least one other step. in some examples, only CIRs which may show promising results with respect to the detection of a person may be used for the remainder of the method.

The method may then comprise sampling, by a processor couplable to the transceiver, a preset number of channel input responses, CIRs, wherein the channel input response is an output of the transceiver after the reflected first signal has been received. In some examples, where bins have been used, only CIRs within bins with strong indications of human presence may be sampled and/or used for the remainder of the method. This may reduce the processing power needed for the method.

The method may then comprise calculating, by the processor, a covariance matrix of the sampled channel input responses, wherein the covariance matrix has a preset correlation length S110. This may allow for the processor to begin to organize CIRs for the following steps of the method.

The method may further comprise repeating S110 until the entire sampling time has been sampled S112.

The method may further comprise time aligning each row of the matrix by circular shifting, and by one CIR sample S114. Again, this may allow for the processor to begin to organize CIRs for the following steps of the method.

The method may further comprise calculating, by the processor, a complex sum of each entry of the covariance matrix S116. This may allow for the method to begin the process of determining the power spectral density of the sampled CIRs.

In some examples, the method may further comprise calculating a complex sum of all columns of the matrix, wherein the calculation of the complex sum results in a column array. Again, this may allow for the method to begin the process of determining the power spectral density of the sampled CIRS.

The method may then comprise calculating, by the processor, a power spectral density of each complex sum S118. The skilled person understands how a power spectral density is calculated.

The method may then comprise finding, by the processor, a peak in the power spectral density, wherein the peak is an indication of a person S120. This may allow for the person to be accurately found and/or for a vital sign, such as, for example, the breathing rate and/or the heart rate of the person to be determined and/or determine a distance between the person and the device using the method.

In some examples, the method further comprises sending S122 to a display and/or alarm unit couplable to the processor, a second signal comprising data indicating which bin the peak has been found in S120. This second signal may also comprise visual and/or audio and/or haptic and/or any other suitable data configured to alert the user of the device using the method to the presence of a person and/or a distance from the device to the person.

In some examples, the method may comprise some or all of the following steps:
1. Sampling the signal with number of CIRs/s (number can be between 10 and 250).
2. Select the bin to observe.
3. Decimating by a factor of 10.
4. Calculating the covariance matrix with a correlation length of X seconds, e.g.
X=8s (to estimate heart rate, a shorter correlation length may be used).
   a. Take 8s from the last 60s.
   b. Calculate the covariance of these 8s to the rest of the 60s.
   c. Move the 8s signal by 1 sample and calculate the covariance over the rest of the signal.
   d. Proceed until the entire signal is used as the 8s part for the covariance calculation.
   e. Collect these results in a matrix where in every line the values from the new 8s part are stored. Every column holds the covariance of this 8s part and the corresponding signal.
5. Time align the matrix by circular shifting every line by one sample.
6. Calculate the complex sum of all columns. This results in a column array.
7. Calculate the power spectral density.
8. Find the highest peak in the range of 7 to 50 breaths/s.
9. The highest peak (or the fundamental frequency) is the respiration rate.

The most likely frequency may be identified. In a first approach, this may be the highest peak in the power spectral density. However, sometimes one observes also harmonics of that fundamental frequency, and these higher harmonics, incidentally, could include a peak which is higher than the peak of the fundamental frequency. In this case, one may use the fundamental frequency as most likely estimate.

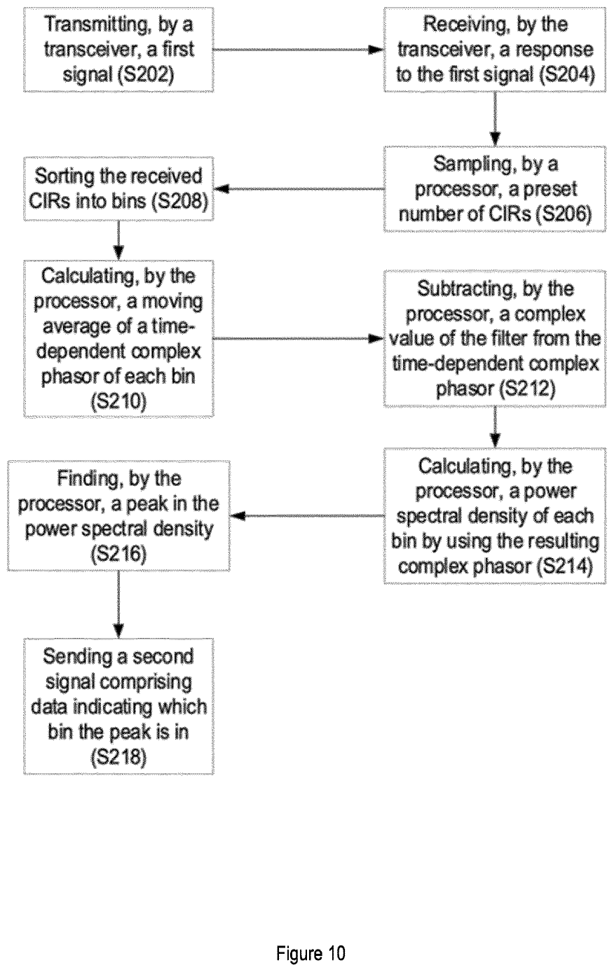

FIG. 10 shows a flow diagram of a method for detecting a person according to some examples as described herein.

In the method of FIG. 10, the method begins by transmitting, by a transceiver, a first signal S202. The first signal may be a UWB signal as described herein. In some examples, a transmitter may be used instead of a transceiver. A response to the first signal may then be received by the transceiver S204. The response may comprise at least a part of the transmitted first signal which has been reflected off of a surface. The surface may be a person, as described herein. In some examples, a receiver may be used instead of a transceiver.

The method may then comprise sampling, by a processor couplable to the transceiver, a preset number of channel input responses, CIRs, wherein the channel input response is an output of the transceiver after the reflected first signal has been received S206.

The processor, in some examples, may then sort each received first signal into a respective bin, wherein each bin represents a distance, or a range of distances, from the transceiver to the surface S208. Examples of bins may be, for example, 1 to 1.99 meters, 2 to 2.99 meters, or any other suitable distance or range. In some examples, the bin may be automatically set by a user of a device which uses the method and/or be a preset bin.

In some examples, the method then comprises observing only a subset of bins. In such a case, only CIRs within bins with strong indications of human presence may be sampled. This may reduce the processing power needed for the method.

The method may then comprise calculating, by the processor, a moving average of a time-dependent complex phasor of each bin S210. This may allow for the processor to determine if a bin and/or CIR comprises an indication of a person and/or an indication of a vital sign of a person.

In some examples, the method may then comprise subtracting a complex value of the filter from the complex phasor S212. This may remove any variables within the bin and/or CIR introduced by the Kalman filter filtering the CIR and/or bin.

The method may then comprise calculating, by the processor, a power spectral density of each bin using the resultant complex phasor S214. The skilled person understands how a power spectral density is calculated.

The method may then comprise finding, by the processor, a peak in the power spectral density, wherein the peak is an indication of a person S216. This may allow for the person to be accurately found and/or for a vital sign, such as, for example, the breathing rate and/or the heart rate of the person to be determined and/or determine a distance between the person and the device using the method.

In some examples, the method further comprises sending S218 to a display and/or alarm unit couplable to the processor, a second signal comprising data indicating which bin the peak has been found in S216. This second signal may also comprise visual and/or audio and/or haptic and/or any other suitable data configured to alert the user of the device using the method to the presence of a person and/or a distance from the device to the person.

In some examples, the method may comprise some or all of the following steps:
1. Sampling the signal with 10 to 250 CIRs/s.
2. Select the bin to observe.
3. Use a Kalman filter to calculate the moving average of the time-dependent complex phasor of the bin. The motion model is uniformly accelerated.
4. Subtract the current complex value of the Kalman filter from the current value of complex phasor of the bin (trend compensation).
5. Calculate the power spectral density of the resulting complex phasor (i.e. the complex phasor of the difference).
6. Find the highest peak in the range of 30 to 180 beats/min (noting that, in some examples, only results in this frequency range may be accepted).
   a. Or: Check the peaks for harmonic frequencies, find the first harmonic (fundamental/base frequency).
   b. Compare to breathing rate and harmonics of breathing rate and decide for the most likely heart rate estimate.
7. The highest peak (or the fundamental frequency) is the respiration rate.
   a. The true peak is searched iteratively in a dichotomous way by calculating spectral lines left and right of the peak in the FFT.

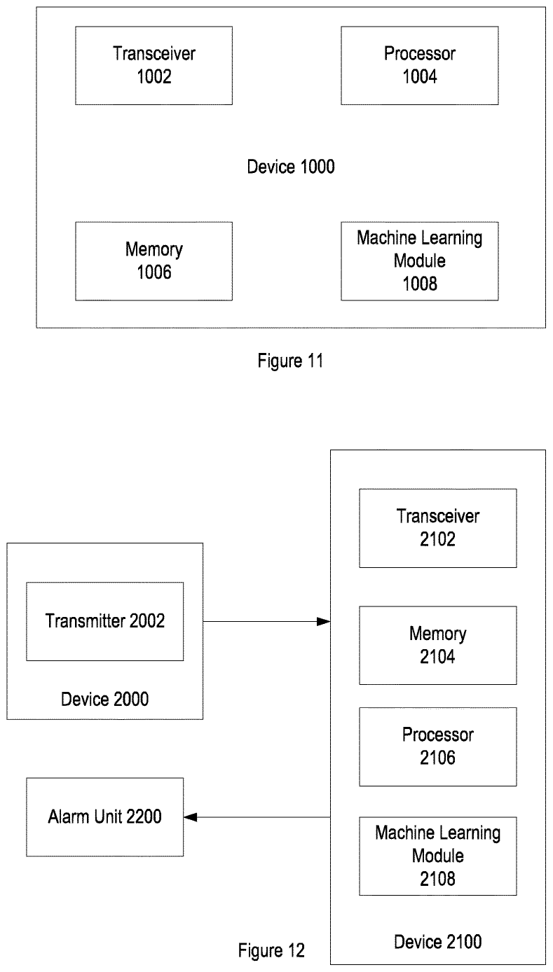

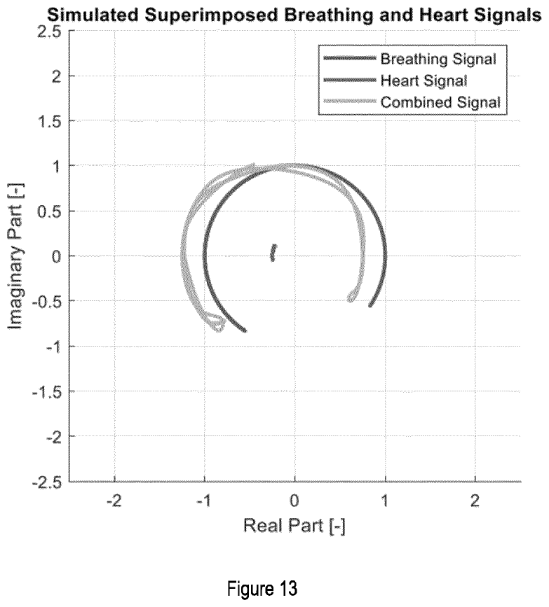

FIG. 11 shows a schematic illustration of a device for detecting a person according to some examples as described herein and FIG. 13 shows a system for detecting a person according to some examples as described herein.

The device 1000 of FIG. 11 comprises a transceiver 1002, a processor 1004, a memory 1006 and a machine learning unit 1008. Each of these features may act and function as described herein. Each of the features may be communicatively coupled to one another, which may allow for communications between each feature to take place. The communication may be via a wired connection and/or a wireless connection. In some examples, the transceiver 1002 may be able to transmit, to an external device, data and/or a signal comprising data indicating that a person has been detected by the methods described herein and/or a distance between the person and the device 1000 using the methods described herein.

The system of FIG. 12 comprises a first device 2000 comprising a transmitter 2002, a second device 2100 comprising a transceiver 2102, a memory 2104, a processor 2106 and a machine learning module 2108, and an alarm unit 2200. Each of these features may act and function as described herein. Each of the features may be communicatively coupled to one another, which may allow for communications between each feature to take place. The communication may be via a wired connection and/or a wireless connection. In some examples, the transceiver 2102 may be able to transmit, to an external device, data and/or a signal comprising data indicating that a person has been detected by the methods described herein and/or a distance between the person and the device 2100 using the methods described herein. In some examples, this external device may be the alarm unit 2200. The alarm unit 2200 may further comprise a display. The alarm unit 2200 may output a visual and/or audio and/or haptic alarm indicating to the user that a person has been detected and/or a vital sign of a person has been detected. In some examples, the alarm unit 2200 may be part of the first device 2000 and/or the second device 2100.

The processors 1004, 2106 and/or memories 1006, 2104 described herein may be entirely in the respective device 1000, 2100, entirely in the cloud, or a combination of the two.

A UWB radar system with two or more antennas may be used in accordance with the methods, devices and systems described herein. At least one of the antennas may be a transmit, Tx, antenna and at least one of the antennas may be a receive, Rx, antenna. The UWB module may be a commercial off-the-shelf UWB system using the IEEE 802.15.4 standard. This may be equivalent to the transmitter 2002 and/or transceivers 1002, 2102 described herein.

At least one of the antennas may be a Through-the-wall Radar configured to detect objects through obstacles, wherein the antenna pattern is chosen such that the crosstalk between Tx and Rx is minimized and their beams are directed towards the person under test.

It may be used 2, 3 or more antennas, for 1D, 2D, 3D and more radar detection to achieve better resolution, calculate Angle-of-Arrival (AoA) and to estimate the body shape.

The processor 1004, 2106 may be configured to identify a human and/or the vitals of the human using a machine learning algorithm stored in the memory 1006, 2104 and/or the machine learning module 1008, 2108. The vital monitoring device 1000, 2000, 2100 may further comprise an alarm unit 2200 configured to receive, from the machine learning module 1008, 2108 and/or device 1000, 2000, 2100, information on the identified object, i.e. person, and generate alert signals.

The complex channel impulse responses, CIRs—estimated at the Rx based on signal sequences sent by the Tx—may be the starting point of the signal processing. In the case of multiple Rx antennas, there may be one CIR, or one stream of consecutive CIRs, per Rx antenna.

The system, in some embodiments, may further comprise a vital monitoring device 1000, 2000, 2100, whether stationary or mobile, configured to detect humans as well as their behavior and vitals based on the sensed parameters received from said sensors using a machine learning algorithm in a machine learning module 1008, 2108. The machine learning module 1008, 2108 may also be capable of analyzing an environment, such as, for example, presence of walls and/or metal structures. The system may further comprise a communicating system 2200 configured to receive alert notifications transmitted from the vital monitoring device.

Embodiments in accordance with the present invention may further provide a vital monitoring device 1000, 2000, 2100 configured to detect humans as well as their vitals. The vital monitoring device 1000, 2000, 2100 may comprise a receiving and transmitting antenna 1002, 2002, 2102 in a form of a radar and/or an Ultra-Wide-Band, UWB, chip or similar chips using radar impulse signals, such as, for example, Frequency Modulated Continuous Waves, FMCW. The vital monitoring device 1000, 2000, 2100 may comprise 2, 3 or more antennas, for 1D, 2D and 3D radar and more detection and to achieve better resolution. The vital monitoring device 1000, 2000, 2100 may further comprise an electronic circuit (PCB) couplable to the receiver and/or the transmitter 2002 and/or the transceiver 1002, 2102, wherein the electronic circuit may comprise (i) a signal pre-processing and post-processing unit configured for optimized signal processing of the received UWB signals, specifically aimed for optimizing the crosstalk generated from the communication between transmitted and received impulse signals, related antennas 1002, 2002, 2102, circuitry board and connectors, and related processing logic thereto; and/or (ii) a frequency converter configured to convert a frequency to the phase measured in a time domain; and/or (iii) signal processing methods for filtering out noise to obtain clear frequency for further analysis. The vital monitoring device 1000, 2000, 2100 may further comprise a machine learning unit, MLU, or machine learning module 1008, 2108, couplable to the electronic circuit and/or processor 1004, 2106 and/or memory 1006, 2104, wherein the machine learning module 1008, 2108 may comprise a processor and/or a memory. The MLU 1008, 2108 may comprise methods for defining the human target distance to the device and/or coordinates and/or time-of-flight, using advanced phase analysis and/or methods used for defining the human heart-rate without breathing and breathing alone and/or methods used for deriving heart-rate from breathing and/or methods used for deriving blood flow in blood vessels and/or methods used for detecting vitals of a moving target or multiple stationary or moving targets in line-of-sight, LOS, and in non-line-of-sight, NLOS-through the obstacles and/or the methods described herein.

In FIG. 13, a heartbeat signal and a breathing signal are superposed. The simulation parameters for each signal may be at least one of:
The angle variation amplitude, which may influence the span of the arc described by the signal;
The magnitude of the complex phasor;
The angular speed of the phasor (not visible in FIG. 13); and
The initial phase angle of the phasor

The angle variation amplitude of the breathing signal may correspond to a movement of 1.7 cm (0.75·π), whereas the heartbeat may have an angle variation amplitude that corresponds to a movement of 2.3 mm (0.1·π).

The shape of the angle variation that is used to model the movement of the phasors may be sinusoidal. The heart-rate may be simulated to be 57 bpm, and the breathing signal 9.18 bpm. These elements are not visible in FIG. 13 but their difference may show up in the combined signal where the heart-rate is modulated on top of the breathing signal.

The initial phases may be different for each phasor and may be chosen arbitrarily. The combination of the four above-mentioned parameters may lead to an offset of the combined signal with respect to the origin. Furthermore, the pattern drawn by the combined curve may have a periodicity which is given by the least common multiplier of the angular speeds of the phasors.

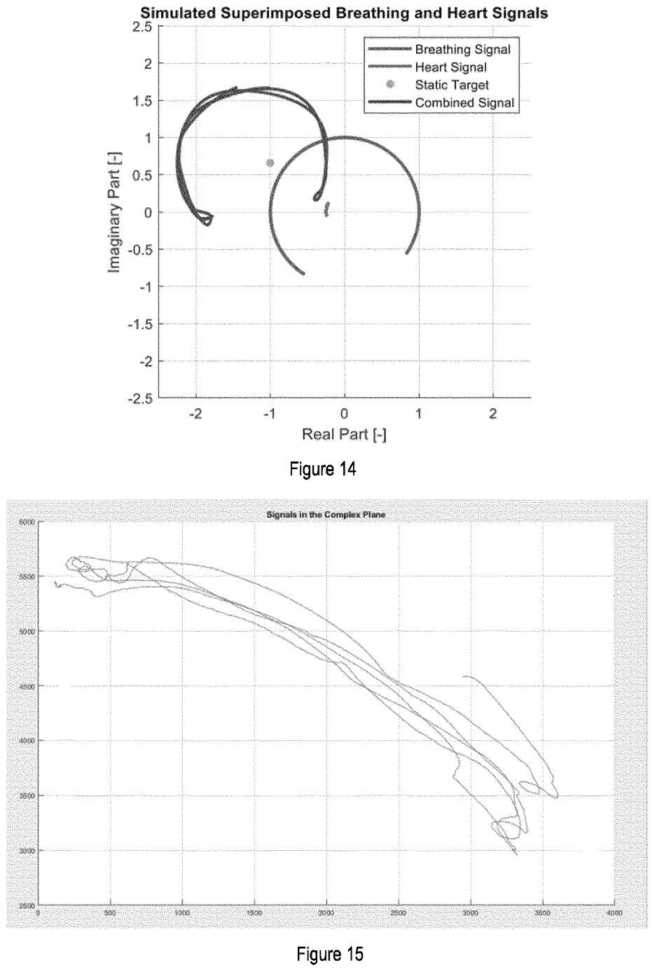

From this simulation, the case could be extended by an additional static phasor representing a static target in the bin. One phasor may be enough to model any linear combination of phasors corresponding to static targets. This may make it difficult to resolve multiple static components. The offset could be removed if the parasitic signals are static. This may correspond to a scenario where the subject is not moving at all, except their chest due to breathing and heartbeat. The parasitic targets may be any parts of the body or element of furniture lying in the same range bin as the moving parts. Such a scenario is shown in FIG. 14.

Such an offset compensation could be helpful for the breathing rate extraction so that only the phase could be extracted and directly processed. The observed signals may not have a big enough span to easily estimate the center of the arc. In addition to that, the curve drawn by one breathing in and breathing out cycle may show a hysteresis effect. This may be due to different uses of the muscles in the chest area which are used to inflate and deflate the lungs. This could indicate that a part of the signal also penetrates the body which makes for a more complex target model.

From the signals shown in FIGS. 13 and 14, one can imagine how chaotic movement from a body part may heavily distort the arc shaped signals shown.
1. Due to the amplitude of the movements being easily above one wavelength of the carrier signal, the induced phase angle modulation may be in the range of several full rotations; and
2. The change in orientation in the body part may also mean a magnitude modulation.

Such body part movements may often have no predictable or periodic structures, and they can be thought of as noise that may be added up to the true vital signs signal. Unpredictable movements may therefore be very challenging to compensate for, prior to the extraction of the time varying signals that are of interest, namely the breathing and heartbeat signals. As explained with the static parasitic targets, also in such a case, there may be no way to observe each moving part separately.

FIG. 15 shows a recording of a breathing person, wherein the arc shaped curve is due to the breathing, and the curls are due to the heartbeat.

The signals that may be extracted can be very close to each other in the spectrum. For an average person, the breathing rate may range from 7 to 15 bpm or 0.1-0.25 Hz, whereas the typical heart-rate may be located between 50 and 200 bpm (0.83-3.3 Hz). With a frame extraction rate of 200 fps, the frequency resolution of an FFT may be dependent solely on the observation time. The numbers above may show that a sub-Hertz resolution is needed to separate the breathing signals from the heartbeat signal.

Furthermore, with such ranges in the heartbeat and breathing, the chances of harmonics in the breathing signal interfering with the heart-rate signal may be non-negligible. This may be an aggravating case for the heart-rate extraction.

Since the heart-rate may be combined with the breathing signal, the challenge may be to isolate the heart-rate. In the approach mentioned herein, the trend of the signal in the complex plane may be estimated with a Kalman filter, although the skilled person understands that any other suitable filtering technique can be used. The filter may consider position and/or velocity and/or acceleration in the x and/or y components, which makes for 6 states.

After the phase extraction, a DC blocking filter may be applied to the signal to remove any strong DC components in the frequency domain. The reason for this is that the breathing frequency may be located close to the DC bin. In presence of a strong static component, the breathing rate peak and the DC peak may melt together and hinder the breathing rate estimation.

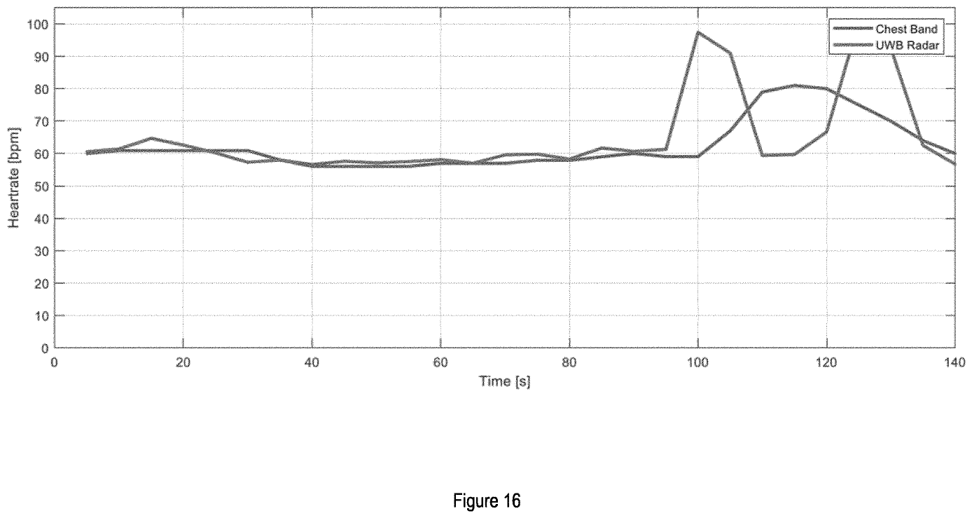

The accuracy of the heart-rate measurement may be verified with a chest belt. Both the radar and the chest belt measurement may be started simultaneously, and the outputs may be captured by a screen recording showing the radar heart-rate estimation as well as the chest belt heart-rate estimation. The results of such a method are shown in FIG. 16.

In the example of FIG. 16, up to 95 seconds, the measurement was performed with ideal posture of the subject and close to no movement. From 95 seconds to the end of the readings, the subject started moving their arms around to mimic natural movements. As it its depicted in the graph, this shows to be very challenging for the algorithm. As stated before, the phase shifts induced by arm movements may mask the vital signs. To overcome this problem, the beam of the antenna may, in some examples, have a much higher directivity to focus the measurement on a smaller area.

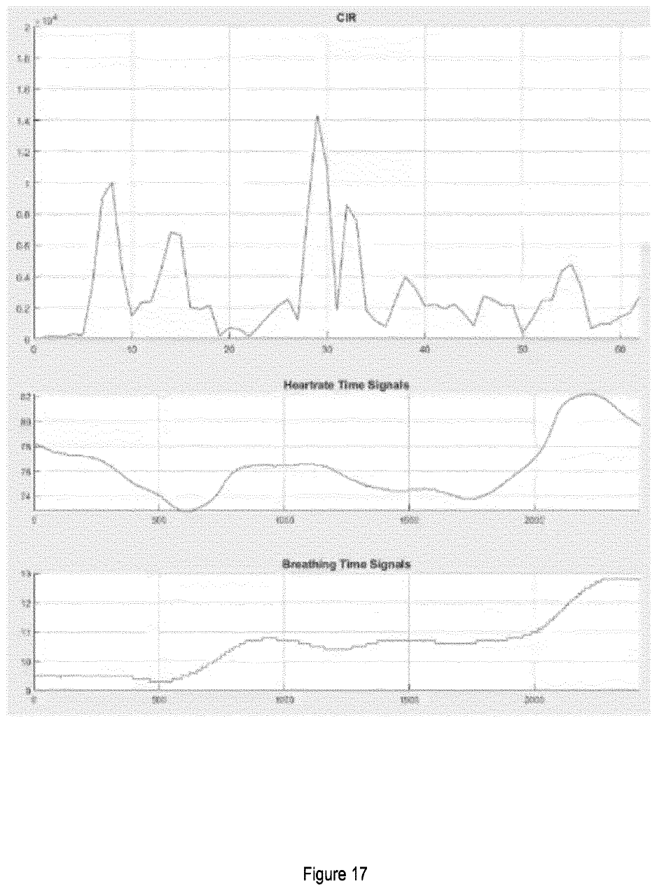

On the other hand, the accuracy of the breathing rate extraction algorithm may be assessed by breathing a given rate with a metronome giving a rhythm of 15 bpm. The result is visible in FIG. 17 between samples 950 and 2000. The x-axis shows the sample index, it can be converted to seconds with the following equation:

$$t = \frac{{\#}_{sample}}{200} * 8 [ s ]$$

In the above, t is the time, #sample is the number of the specific sample which is used to calculate one specific time instant, 200 is the denominator as there are 200 samples per second, although this can be altered based on the sampling rate, and 8 is used as this is the correlation length in seconds, but this can also be altered based on the scenario.

In addition, the system may further comprise computing devices and methods configured to analyze data points received from sensors and/or from embedded processing nodes to build predictive modeling generated therefrom on the remote cloud processing nodes, with the aim to detect early signs of risks of vitals failure.

Human detection may be made by measurements taken from the radar signal determining one or more of a size of the object, a direction, a speed of movement, and one or more vital signs, which may comprise elements for consideration of supervised machine learning, ML, algorithm within the machine learning module 1008, 2108 to identify that the object is a human. The purpose of the first machine learning is to classify the age of the person. It is well known that the breathing rate of a person varies with age. For example, the breathing rate of a child at birth to 1 year old is 30-60 breaths per minute and reduces with age.

However, based solely on breathing rate detection, is not possible to reliably detect whether it is a child. Therefore, machine learning methods may be used, (it can also be edgeML), originally trained on synthetic data of the age and breathing and heart-rate available from medical studies, combined with the reflecting energy of a person (measured from the data obtained from signal which has been reflected off an object).

For adults, a number of datasets is collected. For child presence detection vitals detection, the breathing dolls are used.

Secondary machine learning methods within the machine learning module 1008, 2108 may be applied to identify the well-being of the people or their intentions by their movement patterns and vital signs.

It is also known that from the breathing rate combined with heart-rate it is possible to estimate the physiological state, such as pain, emotional stress, heart attack. Following the first ML principle above, the second machine learning method is applied to detect if the person is becoming incapacitated. This machine learning module is trained based on the available datasets of the heart-rate and breathing rate and heart-rate and breathing variability combined with the algorithms mentioned above (using detecting body parts of a person for body micro-movements). A combination of body micromovements combined with the breathing rate and heart-rate and its variability allows to predict a few seconds prior if the person is becoming incapacitated. And as an opposite, it can also allow to detect a type of person, practicing high athletic activities, for example.

Tertiary machine learning methods within the machine learning module 1008, 2108 may be applied in combination of other sensors in a form of sensor fusion to trigger the alerts.

The third machine learning method is a combination of outputs or datasets received (by outputs the breathing rate etc. is meant) from the UWB sensor, combined with the outputs or datasets received from cameras that can be used for the eye position tracking movement, as an example, which will improve the predictive models of detecting people before they become incapacitated (heart attack, epileptic fit, etc.) and forming sensor fusion platform around human vitals and biological signatures. The resulting model of such comprises of collecting and sending data, and processed by local embedded processing nodes, which is more accurate for vitals detection and building predictive models around human behavioral patterns, because it balances the strength of different sensors combined.

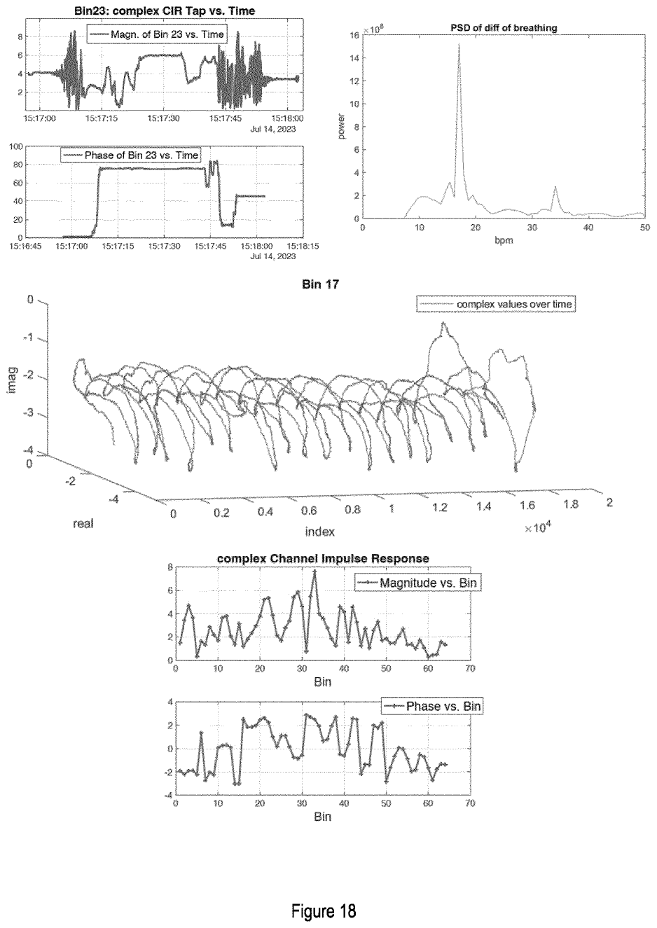

FIG. 18 shows graphs of the results from the method according to some examples as described herein.

Bins 23 and 17 are mentioned in FIG. 18, but the skilled person understands that these are just examples for the figures, and any bin can be used. A bin, as mentioned above and described herein, may relate to a range of distances, or a single distance, between the receiver/transceiver, and the object.

The magnitude and the phase of the complex CIR is shown in relation to time. Here, it can be seen that the received signal has a magnitude and phase dependent on where the reflector is in relation to the device and/or transmitter and/or transceiver and/or receiver. The device and/or an electronic circuit coupled to the receiver and/or transceiver may be able to determine the magnitude and phase of the received signal.

A complex value of the received signal in relation to bin 17 is also shown. Here, a time-dependent complex phasor is shown, as is mentioned herein. Furthermore, the term “index” may relate to a sampling index of the signal, and may relate to the time axis and/or the time over which readings and/or signals are received. It can be seen that there is a repetition to the received signal, indicating the presence of a person as they breathe and/or as their heart beats.

Furthermore, a result of the power spectral density calculation is shown. Here, it relates to the respiration rate, but the skilled person understands that the same principle applies to the heart rate. The highest peak of the power spectral density calculation may indicate the breathing rate of the person being detected by the device/system.

Additionally, two graphs relating to the magnitude and phase of the received signal with respect to bins are shown. It can be seen that some of the bins have a higher magnitude and/or phase. In this case, the method and/or device and/or system described herein may only sample the bins with a magnitude and/or phase above a user-defined and/or predetermined threshold, as these bins may be more indicative of the presence of a person.

No doubt many other effective alternatives will occur to the skilled person. It will be understood that the invention is not limited to the described embodiments and encompasses modifications apparent to those skilled in the art and lying within the scope of the claims appended hereto.

1. A method for detecting a presence of one or more living objects by measuring one or more vital signs of the one or more living objects, the method comprising:
transmitting, by a transceiver, a first signal;
receiving, by the transceiver, a response to the first signal from any object in an environment from which at least a part of the transmitted first signal has been reflected off from the object towards the transceiver;
translating, by one or more algorithms, the response into raw data;
selecting, by the one or more algorithms, a bin by processing and filtering the raw data based on a reflected energy received by the transceiver via the response, corresponding to a distance of a living object to the transceiver; sampling results, by the one or more algorithms, from the bin obtained via the selection;
analyzing, by the one or more algorithms, the results to determine the presence and one or more vital signs of any living object from which the transmitted first signal has been reflected off towards the transceiver;
outputting one or more vital signs parameters corresponding to the determined one or more vital signs.

2. A method as claimed in claim 1, further comprising analyzing the one or more vital signs parameters to determine a physiological state of the one or more living objects.

3. A method as claimed in claim 1, further comprising, classifying a type of the one or more living objects via a combination of an amplitude of the reflected energy, an amplitude of an oscillating movement of the one or more living objects and the one or more vital signs.

4. A method as claimed in claim 3, wherein the type comprises a human and/or an animal.

5. A method as claimed in claim 1, further comprising classifying an age of the one or more living objects by determining a size of the object via a combination of an amplitude of the reflected energy and the one or more vital signs.

6. A method as claimed in claim 1, wherein the signal is obtained from an ultra-wideband, UWB, device and/or from a plurality of UWB devices forming a distributed radar system.

7. A method as claimed in claim 1, wherein the one or more vital signs comprise one or more of a breathing rate, a heart-rate, a breathing rate variability and a heart-rate variability.

8. A method as claimed in claim 1, further comprising combining the one or more vital signs parameters with one or more datasets obtained from one or more tertiary sensors, such as one or more video cameras and/or one or more biometrical sensors, to form a sensor fusion platform.

9. A method as claimed in claim 1, wherein the selecting comprises:
calculating channel input responses, CIRs, based on the received response, wherein each of the CIRs is an output of the transceiver after the reflected first signal has been received by the transceiver;
calculating the selected CIRs by applying a breathing extraction algorithm and/or a heart-rate extraction algorithm.

10. A method as claimed in claim 9, wherein applying the breathing extraction algorithm and/or the heart-rate extraction algorithm comprises:
calculating a covariance matrix of the subset of CIRs;
calculating a complex sum of all columns of the covariance matrix to obtain a column array;
calculating a power spectral density of each complex sum;
finding a highest peak in the power spectral density; and
analyzing the highest peak to determine the presence detection and one or more vital signs.

11. The method of claim 9, wherein a preset number of CIRs per second is determined by the one or more algorithms.

12. A method as claimed in claim 1, wherein the part of the transmitted first signal is received by the transceiver due to propagation of the transmitted first signal.

13. A method as claimed in claim 1, wherein the received response is provided to a frequency converter configured to covert a frequency to a phase measured in a time domain.

14. A method as claimed in claim 1, further comprising calculating a distance between the transceiver and the object based on a time of flight of the first signal, wherein the time of flight is a time between the transmission and reception of the first signal.

15. A method as claimed in claim 9, further comprising sorting each CIR into a respective bin, wherein each bin represents a distance, or a range of distances, from the transceiver to the object.

16. A method as claimed in claim 10, further comprising, based on the finding of the peak in the power spectral density, sending, to a display and/or alarm unit, a second signal comprising data indicating which bin the peak has been found in.

17. A method as claimed in claim 10, wherein the calculation of the covariance matrix is based on selecting a first period of time which is preferably 8 seconds or less.

18. A method as claimed in claim 10, further comprising storing the covariance matrix in a memory, wherein each row of the matrix comprises a respective covariance.

19. A method as claimed in claim 10, wherein the calculation of the covariance matrix is based on selecting a first period of time which is preferably 60 seconds or less.

20. A method as claimed in claim 9, wherein the subset of CIRs is 10% of the total number of CIRs

21. A method as claimed in claim 10, wherein the method further comprises:
checking if any of the peaks of the power spectral density calculation are harmonic frequencies, and
finding a fundamental frequency.

22. A method as claimed in claim 10, wherein the highest peak of the power spectral density represents a vital signs parameter, in particular a respiration rate of a living object.

23. A method as claimed in claim 1, wherein the selecting comprises:
sampling a preset number of channel input responses, CIRs, wherein the channel input response is an output of the transceiver after the reflected first signal has been received by the transceiver;
sorting each CIR into a respective bin, wherein each bin represents a distance, or a range of distances, from the transceiver to the object;
calculating a moving average of a time-dependent complex phasor of each bin using a filter;
subtracting a current complex value of the filter from a current value of the time-dependent complex phasor;
calculating a power spectral density of a complex phasor of a difference between the current complex value of the filter and the current value of the time-dependent complex phasor; and
finding a peak in the power spectral density.

24. The method of claim 23, further comprising observing a subset of bins and perform the method from the calculation of the moving average step onwards using only the subset of bins.

25. The method of claim 23, wherein the filter comprises a Kalman filter.

26. The method of claim 25, wherein the Kalman filter is configured to use a uniform acceleration motion model.

27. The method of claim 23, wherein the finding of the peak comprises finding the highest peak in a range of 30 to 180 Hertz.

28. The method of claim 23, wherein the method further comprises checking if any of the peaks of the power spectral density calculation are harmonic frequencies, and finding a fundamental frequency.

29. The method of claim 23, wherein a peak of the power spectral density represents a vital signs parameter, in particular a heart rate of a living object.

30. The method of claim 23, wherein finding the peak comprises iteratively searching spectral lines on either side of a peak in a Fourier Transform of the power spectral density in a dichotomous manner.

31. The method of claim 30, wherein a Goertzel filter is used to find the peak.

32. The method of claim 23, wherein the number of CIRs is between 10 and 250 CIRs per second.

33. The method of claim 23, further comprising, based on the finding of the peak in the power spectral density, sending, to a display and/or alarm unit, a second signal comprising data indicating which bin the peak has been found in.

34. The method of claim 23, wherein only a subset of CIRs are sorted, and wherein the subset comprises 10% of the total number of CIRs.

35. The method of claim 23, wherein the part of the transmitted first signal is received by the transceiver due to propagation of the transmitted first signal.

36. A method as claimed in claim 1, further comprising:
obtaining, from one or more sensors, in particular one or more cameras and/or one or more biological sensors, data relative to the one or more living objects, and
fusing the one or more vital signs parameters with the data obtained from the one or more sensors to predict a physiological state of the one or more living objects.

37. A device comprising:
a transceiver configured to transmit a first signal and to receive a response to the first signal,
wherein the response comprises at least a part of the transmitted first signal which has been reflected off of an object; and
a computer readable storage medium comprising instructions which, when executed by a processor, cause the processor to carry out the method of claim 1.
